(19) United States
(12) Patent Application Publication
(10) Pub. No.: US 2025/0161680 A1
(43) Pub. Date: May 22, 2025

(54) Systems and Methods for Electro-Medical Stimulation with Stimulatory and Rest Treatment Phases

(71) Applicant: International Rehabilitative Sciences, Inc., Vancouver, WA (US)

(72) Inventors: Randy Alan Murphy, Vancouver, WA (US); William J. Carroll, Vancouver, WA (US); Sydney J. Newkirk, Canby, OR (US)

(21) Appl. No.: 18/913,687

(22) Filed: Oct. 11, 2024

Related U.S. Application Data
(60) Provisional application No. 63/602,144, filed on Nov. 22, 2023.

(51) Int. Cl. A61N 1/36; A61N 1/02; A61N 1/04

(52) U.S. Cl. CPC A61N 1/36034; A61N 1/025; A61N 1/0452; A61N 1/0456; A61N 1/36021

(57) ABSTRACT

An example apparatus for electro-medical stimulation includes a current generator to generate a sine wave output treatment signal, and a processor coupled to the current generator to modulate a frequency and a power of the sine wave output treatment signal over a cycle of stimulation treatment to produce periods of action potential for a stimulatory treatment phase and periods of non-action potential for a rest treatment phase. The cycle of stimulation treatment includes a first time period followed by a second time period, and during the first time period, the cycle of stimulation treatment is in a period of action potential for the stimulatory treatment phase, and during the second time period, the cycle of stimulation treatment is in a period of non-action potential for the rest treatment phase.

### CROSS REFERENCE TO RELATED APPLICATION

The present disclosure claims priority to U.S. provisional application No. 63/602,144, filed on Nov. 22, 2023, the entire contents of which are herein incorporated by reference.

### FIELD

The present disclosure is generally related to electro-medical stimulation, and more particularly to, modulation of a frequency and a power of a sine wave output treatment signal by an apparatus over a cycle of stimulation treatment to produce periods of action potential for a stimulatory treatment phase and periods of non-action potential for a rest treatment phase.

### BACKGROUND

Numerous electrical devices are commonly used in medicine today, including Transcutaneous Electrical Nerve Stimulators (TENS) used in physical therapy for temporary pain control and Neuromuscular Electrical Stimulators (NMES) for muscle stimulation.

An example therapy session includes treating a patient with muscle stimulation (“MS”) therapy. MS therapy sessions can be used when a patient has acute pain and/or dis-use atrophy. Administering therapy sessions are often time consuming for the patient, and may not provide adequate treatment. Such administration is also often uncomfortable for patients, because a physician may have to change the position of electrodes between therapy sessions.

### SUMMARY

Within examples, an apparatus for electro-medical stimulation is described, and the apparatus comprises a current generator to generate a sine wave output treatment signal, and a processor coupled to the current generator to modulate a frequency and a power of the sine wave output treatment signal over a cycle of stimulation treatment. This treatment will produce periods of action potential for a stimulatory treatment phase and periods of non-action potential for a rest treatment phase. The cycle of stimulation treatment includes a first time period followed by a second time period. During the first time period, the processor implements a reducing frequency modulation coupled with an increasing power modulation, and a reduction of frequency occurs at a rate less than an increase in power. During the second time period, the processor implements a reducing power modulation coupled with an increasing frequency modulation, and a reduction of power occurs at a rate less than an increase in frequency.

In another example, a method of electro-medical stimulation applied by an apparatus is described. The method comprises generating a sine wave output treatment signal, and modulating a frequency and a power of the sine wave output treatment signal over a cycle of stimulation treatment to produce periods of action potential for a stimulatory treatment phase and periods of non-action potential for a rest treatment phase. The cycle of stimulation treatment includes a first time period followed by a second time period. During the first time period, the method includes implementing a reducing frequency modulation coupled with an increasing power modulation, and a reduction of frequency occurs at a rate less than an increase in power. During the second time period, the method includes implementing a reducing power modulation coupled with an increasing frequency modulation, and a reduction of power occurs at a rate less than an increase in frequency.

In another example, an apparatus for electro-medical stimulation is described. The apparatus comprises a current generator to generate a sine wave output treatment signal, and a processor coupled to the current generator to modulate a frequency and a power of the sine wave output treatment signal over a cycle of stimulation treatment to produce periods of action potential for a stimulatory treatment phase and periods of non-action potential for a rest treatment phase. The cycle of stimulation treatment includes a first time period followed by a second time period. During the first time period, the processor implements a first frequency modulation coupled with a first increasing power modulation so as to cause a muscle contraction level for the stimulatory treatment phase. During the second time period, the processor implements a second frequency modulation higher than the first frequency modulation with a second increasing power modulation higher than the first increasing power modulation so as to be below the muscle contraction level for the rest treatment phase.

These as well as other aspects, advantages, and alternatives will become apparent to those of ordinary skill in the art by reading the following detailed description, with reference where appropriate to the accompanying drawings.

### BRIEF DESCRIPTION OF THE DRAWINGS

Many aspects of the disclosure can be understood with reference to the following drawings. The components in the drawings are not necessarily to scale. Moreover, in the drawings, like reference numerals designate corresponding parts throughout the several views.

FIG. 1 is a block diagram illustrating an example of an apparatus for electro-medical stimulation, according to an example implementation.

FIGS. 2A-2B are schematic diagrams illustrating some circuit components of the apparatus, according to an example implementation.

FIG. 3 illustrates an example of the electrodes, according to an example implementation.

FIG. 4 is a graph illustrating an example of variable stimulation therapy, according to an example implementation.

FIG. 5 is a graph illustrating an example intensity per time operation of the apparatus for variable stimulation therapy, according to an example operation.

FIG. 6 is a graph illustrating another example intensity per time operation of the apparatus for variable stimulation therapy, according to an example operation.

FIG. 7 is a graph illustrating power versus time for application of therapy to stimulate different nerves, according to an example implementation.

FIG. 8 is a graph illustrating intensity (e.g., amplitude or power) over time with variations in frequency, according to an example implementation.

FIG. 9 shows a flowchart of an example of a method of electro-medical stimulation applied by an apparatus, according to an example embodiment.

FIG. 10 shows a flowchart of another example of a method of electro-medical stimulation applied by an apparatus, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Within examples, muscle stimulation for therapy or pain treatment is described in which a stimulation cycle of a treatment phase followed by a rest phase is applied. Typically, when muscle stimulation occurs, in particular below or around about 1 KHz, as frequency is increased an intensity is increased. At frequencies higher than 1 KHz, even though stimulation is applied, a density applied to a target area decreases due to being spread out over a larger area. As a result, intensity (power) is increased simultaneously with frequency.

Within examples herein, a muscle stimulation therapy is described that includes modulation of both frequency and intensity (or power) according to a cycle of stimulation treatment to provide a stimulatory treatment phase followed by a rest treatment phase. During each phase, both of the frequency and power are modulated independently and simultaneously. In one example, therapy includes reducing frequency coupled with increasing power followed by reducing power coupled with increasing frequency. In another example, therapy includes low frequency and low power followed by high frequency and high power. Using example frequency and power generation methods described herein enables muscle stimulation to be more effective over time.

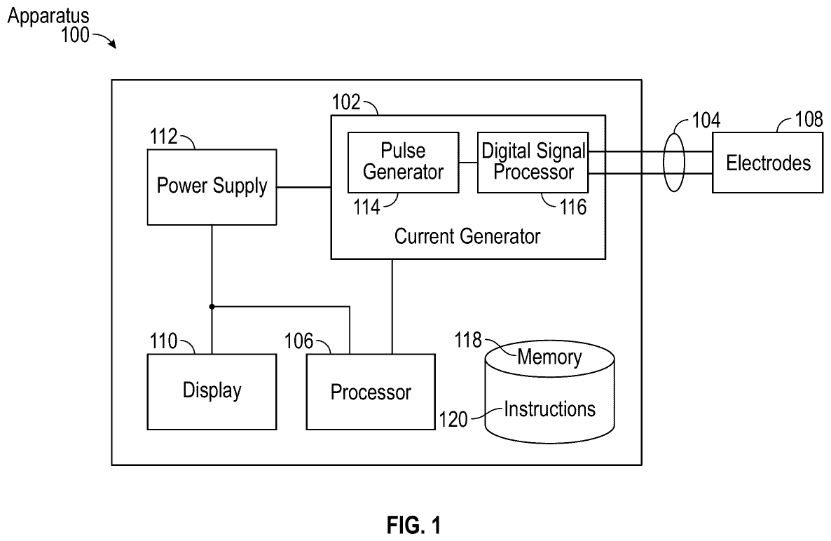

FIG. 1 is a block diagram illustrating an example of an apparatus 100 for electro-medical stimulation, according to an example implementation. The apparatus 100 includes a current generator 102 to generate a sine wave output treatment signal 104, and a processor 106 coupled to the current generator 102 to modulate a frequency and a power of the sine wave output treatment signal 104 over a cycle of stimulation treatment. The apparatus 100 also includes multiple electrodes 108 coupled to the current generator 102 to receive the sine wave output treatment signal 104 for application of the stimulation treatment to a patient.

In addition, the apparatus 100 includes a display 110 coupled to the processor 106, and the display 110 illustrates control and setting information, for example. The display 110 includes a touchscreen display, in one example, configured to receive inputs. The apparatus 100 also includes a power supply 112 coupled to the current generator 102, the processor 106, and the display 110 to provide power for operation of components of the apparatus 100. The power supply 112 includes a battery or an AC input, for example.

The current generator 102 includes a pulse generator 114 that generates digital signal pulses and a digital signal processor 116 coupled to the pulse generator that processes the digital signal pulses to approximate a sine-wave-like output waveform, for example, for the sine wave output treatment signal 104. For example, the output of the pulse generator 114 is a sinewave, pseudo sinewave, or some sine-wave-like continuous waveform that is in-phase. The digital signal processor 116 then transmits the sine-wave-like output waveform as the sine wave output treatment signal 104.

The pulse generator 114 generates individual pulses of differing widths and resultant amplitudes. In some examples, the pulse width is set at 210 microseconds, but can range from 20-600 microseconds. Within examples, a range of output of the sine wave output treatment signal 104 is about 0-50 volts per circuit, depending on the patient's needs for pain treatment.

The processor 106 may be or include a field-programmable gate array (FPGA) used to shape multiple pulsatile waveforms to approximate the output of a sine-wave generator instead of or in addition to a digital signal processor. The FPGA is an integrated circuit that can be programmed in the field after it is manufactured and allows its user to adjust the circuit output as desired. Thus, in an alternative example, the processor 106 may be replaced with the FPGA. An FPGA device can allow for complex digital signal processing applications such as finite impulse response filters, forward error correction, modulation-demodulation, encryption and applications.

In addition, within examples, the processor 106 and the digital signal processor 116 are combined as one microprocessor to perform functions of the apparatus 100. To do so, the apparatus 100 includes memory 118 with instructions 120 executable by the processor 106 to perform functions described herein of the apparatus 100.

An example functionality of the apparatus 100 provides electrically isolated channels delivering various sine wave output treatment signals to the electrodes 108 capable of independently treating separate muscle groups.

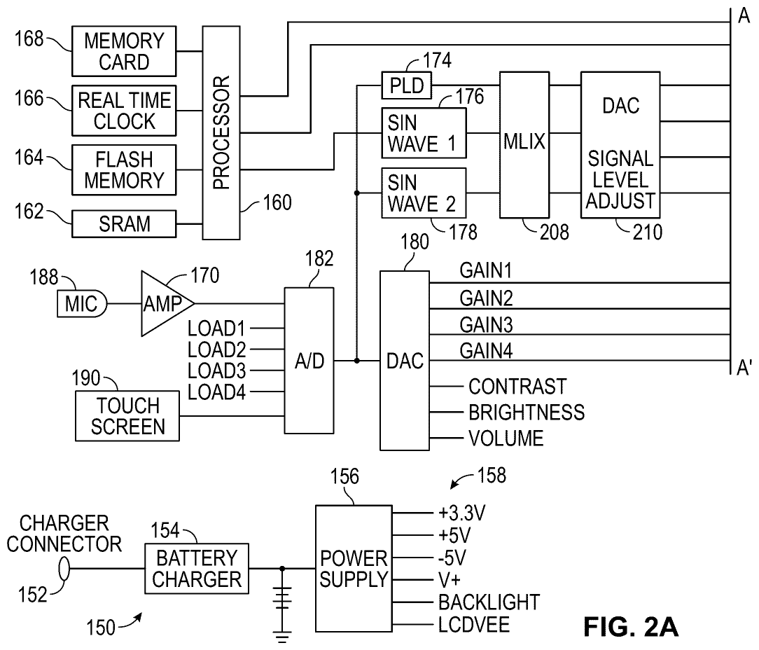

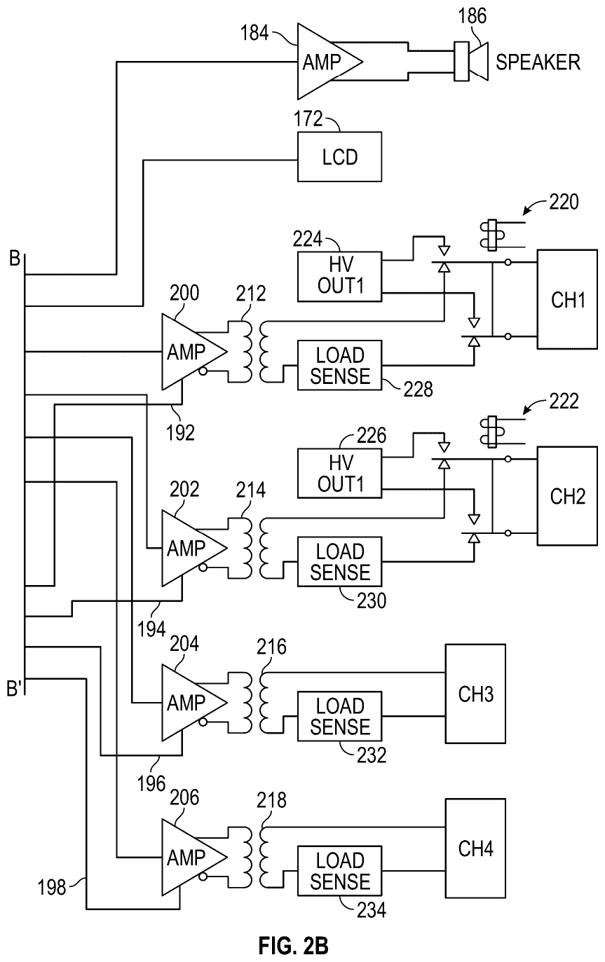

FIGS. 2A-2B are schematic diagrams illustrating some circuit components of the apparatus 100, according to an example implementation. A power supply circuit 150 includes a charger connector 152 in communication with a battery charger 154, which is in communication with a power supply 156. The power supply circuit 150 provides a number of outputs 158 that provide power to other portions of the apparatus 100.

The circuit components also include a processor 160 in communication with static RAM 162, flash memory 164, a real-time clock 166, and a memory card 168. The processor 160 is in communication with an amplifier 170 that controls a liquid crystal display 172, a programmable logic device 174, sine wave generators 176 and 178, a digital to analog converter 180 and an analog to digital converter 182. The processor 160 is also in communication with an amplifier 184 that controls a speaker 186. The analog to digital converter 182 is in communication with a microphone 188 through the amplifier 170 and a touch screen 190. The digital to analog converter 180 provides an output gain 192, 194, 196, and 198 to four channels. The processor 160 controls the digital to analog converter 180 to output a predetermined maximum voltage on those outputs. The outputs 192, 194, 196, and 198 provide input for amplifiers 200, 202, 204 and 206, respectively.

The processor 160 also communicates with the programmable logic device 174 and the sine wave generators 176 and 178 which are multiplexed by a multiplexer 208 to a digital to analog converter 210. The digital to analog converter 210 adjusts the signal level of the amplifiers 200, 202, 204 and 206. The amplifiers 200, 202, 204 and 206 communicate through transformers 212, 214, 216 and 218, respectively. Outputs of the transformers 216 and 218 are provided directly to output of channels three and four, respectively. However, outputs of transformers 212 and 214 are switched through switches 220 and 222 to output channels one and two, respectively. The switches 220 and 222 are solenoids which activate dual bar switches to select the outputs from the transformers 212 and 214 from high voltage outputs 224 and 226. The circuit also includes load sensing devices 228, 230, 232 and 234 which sense the load of corresponding channels one through four, respectively.

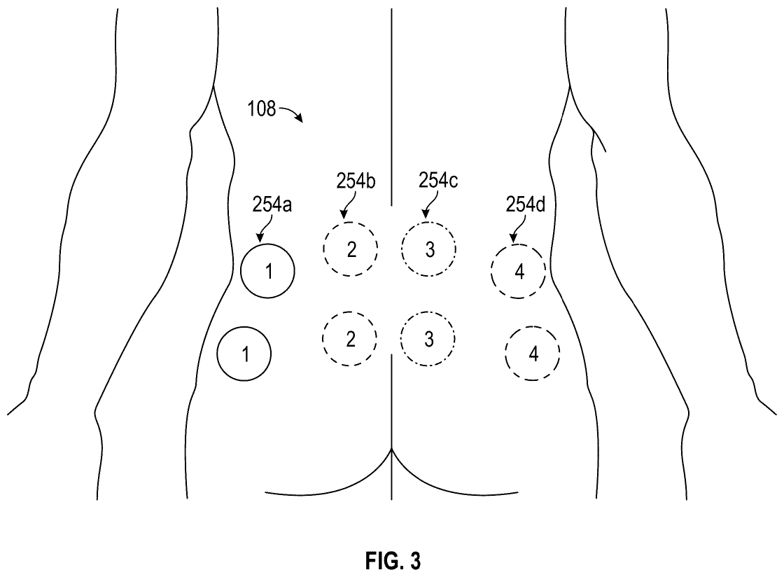

As mentioned, the apparatus 100 provides electrically isolated channels delivering various sine wave output treatment signals to the electrodes 108 capable of independently treating separate muscle groups. FIG. 3 illustrates an example of the electrodes 108, according to an example implementation. The electrodes 108 are shown positioned on a lower back of an individual. The electrodes 108 includes multiple electrode pairs 254a-d and each pair is positioned across an area of the lower back. Any number of electrode pairs can be used depending on an application of therapy.

In one example operation of the apparatus 100, a patient first powers up the apparatus 100 and selects a mode of operation. Various modes of operation exist, such as a normal mode operation in which all four channels act synchronously providing stimulation pulse trains at the same time, although intensities of each channel are independently controlled. This mode of operation allows the patient to independently treat up to four separate muscle groups simultaneously. If the patient desires, an additional level of control for certain situations has been provided, in which channels 1 and 2 are operated asynchronously with channels 3 and 4. Thus, when channels 1 and 2 are stimulating the muscles, channels 3 and 4 are off, and when channels 1 and 2 are off, channels 3 and 4 are stimulating the muscles. The set on and off times are the same for all four channels in the normal mode.

For patient treatment, as a frequency increases (e.g., such as above 1000 Hz), a stimulation threshold increases and a greater intensity (amplitude) is needed to cause an action potential in a target treatment area. Decreasing intensity or increasing frequency thus places the patient below the stimulation threshold and produces a “rest” time for muscle stimulation.

Within examples herein, the apparatus 100 is operated to provide variable stimulation therapy to the patient by modulating frequency and amplitude to produce periods of action potentials (e.g., stimulation periods) and non-action potential periods (e.g., non-stimulation/rest periods).

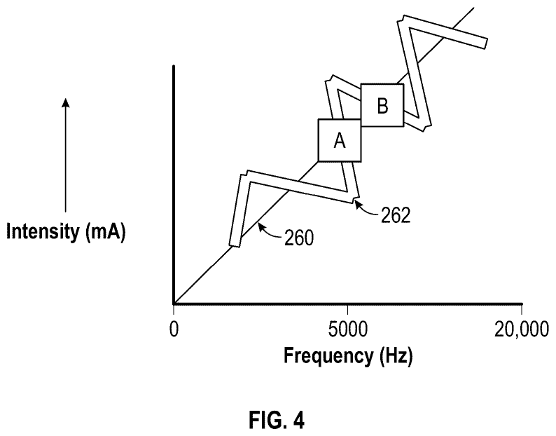

FIG. 4 is a graph illustrating an example of variable stimulation therapy, according to an example implementation. In FIG. 4, a stimulation threshold line 260 is shown to illustrate intensity (mA) and frequency (Hz) combinations above which a patient will feel the stimulation. The apparatus 100 thus produces periods of action potential for a stimulatory treatment phase, and below which the patient will not feel the stimulation and the apparatus 100 thus produces non-action potential for a rest treatment phase. In FIG. 4, a modulation line 262 is also shown to illustrate an example variable stimulation therapy operation of the apparatus 100.

In an example variable stimulation therapy operation of the apparatus 100, the processor 106 modulates a frequency and a power of the sine wave output treatment signal 104 over a cycle of stimulation treatment to produce periods of action potential for a stimulatory treatment phase and periods of non-action potential for a rest treatment phase, and the cycle of stimulation treatment includes a first time period (referenced as “A” in FIG. 4) followed by a second time period (references as “B” in FIG. 4). During the first time period, the processor 106 implements a reducing frequency modulation coupled with an increasing power modulation, and a reduction of frequency occurs at a rate less than an increase in power. Thus, during the time period “A”, the frequency is reduced, shown as reducing from about 5.5 KHz to 5 KHz, and the power is increased, such as increased from 20 ma to 40 mA. During the second time period, the processor 106 implements a reducing power modulation coupled with an increasing frequency modulation, and a reduction of power occurs at a rate less than an increase in frequency. Thus, during the time period “B”, the power is reduced, such as from 40 mA to 30 mA, and the frequency is increased, such as from 5 KHz to 7.5 KHz.

The time period “A” thus implements a slowly reducing frequency modulation coupled with a faster increasing intensity modulation, and the time period “B” implements a slowly reducing intensity modulation coupled with a faster increasing frequency modulation. Thus, during the first time period, the processor 106 implements the reducing frequency modulation simultaneously with the increasing power modulation, and during the second time period, the processor 106 implements the reducing power modulation simultaneously with the increasing frequency modulation.

As a result, during the first time period, the cycle of stimulation treatment is in a period of action potential for the stimulatory treatment phase, and during the second time period, the cycle of stimulation treatment is in a period of non-action potential for the rest treatment phase.

In one example, the cycle of stimulation treatment results in overall increases to the frequency and the power of the sine wave output treatment signal, in one example. In another example, when the power decreases and frequency increases (because for higher frequency, more power is needed to obtain a contraction due to current density being spread out further), this cycle may not necessarily result in an overall increase in frequency and power. It simply depends on an amount of increases and an amount of decreases of the power and frequency, respectively.

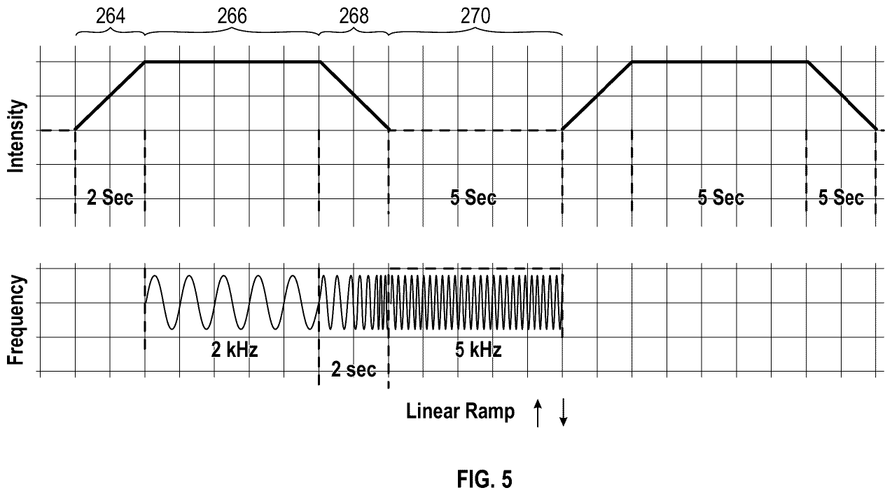

FIG. 5 is a graph illustrating an example intensity per time operation of the apparatus 100 for variable stimulation therapy, according to an example operation. In FIG. 5, the therapy begins with the processor 106 implementing a ramp up of power during a rise time 264 followed by holding power constant during the stimulatory treatment phase 266. During the rise time, the reduction of frequency occurs. For example, the rise time is two seconds during which the power is increased from 0 mA to 20 mA and the frequency is reduced to 2 KHz. The power and frequency are then held constant during the stimulatory treatment phase 266 for five seconds.

Following the stimulatory treatment phase 266, the processor 106 implements a ramp down of power during a fall time 268 followed by holding power constant during the rest treatment phase 270. During the fall time 268, the increase in frequency occurs. For example, the fall time is two seconds, and the frequency is increased to 5 KHz while the power is reduced from 20 mA to 0 mA.

Thus, the therapy implemented by the processor 106 includes the stimulatory treatment phase 266 with high power and low frequency followed by the rest treatment phase 270 with high frequency at low power. The stimulation cycle pattern including the rise time 264, the stimulatory treatment phase 266, the fall time 268, and the rest treatment phase 270 is repeated several times until a desired upper frequency/intensity level is reached, and then a direction will be reversed until the desired lower frequency/intensity level is reached. For example, the processor 106 is configured to repeat the cycle of stimulation treatment until a threshold upper frequency and upper power level is reached, and then the processor 106 is configured to reverse a direction of modulation of the frequency and the power of the sine wave output treatment signal during the first time period (the stimulatory treatment phase 266) and the second time period (the rest treatment phase 270) until a threshold lower frequency and lower power level is reached.

Within examples, the threshold upper frequency and upper power level includes about 10 KHz and about 100 mA. Within examples, the threshold lower frequency and lower power level includes about 1 KHz and about 0 mA.

A lowest base frequency of the sine wave output treatment signal 104 is about 1 KHz and a power (i.e., combination of pulse width and amplitude) will be set to produce a comfortable muscle contraction, for example.

Within examples, the processor 160 modulates the frequency between 1 KHz and 10 KHz, and modulates the power using a current level between 0 to 100 mA. Any combinations of frequencies and power in these ranges are implemented within different examples. Note that power is adjustable by modifying an amplitude of current into a 500 ohm load with 50 Volts, for example. Thus, the processor 160 modulates the power between 0 Watts and 5 Watts using the 500 ohm load with 50 Volts and a range of current between 0 to 100 mA. In the description, power is referenced as being modulated, and it can be assumed that the power is modulated by modulating an amount of current through the circuit as well.

The sine wave output treatment signal 104 is a pre-modulated signal with a beat frequency in a range of 20 beats per second (BPS) to 250 BPS, for example. Using a single circuit with a pre-modulated output signal creates similar effects as if an inferential signal was created with crossing of outputs of two circuits.

In FIG. 5, the stimulatory treatment phase 266 and the rest treatment phase 270 are described as being the same amounts, but the amounts can vary. A length of the stimulatory treatment phase 266 is between about a minimum of 5 seconds to a maximum of 15 seconds. A length of the rest treatment phase 270 is between about a minimum of 5 seconds to a maximum of 30 seconds. The rise time 264 and the fall time 268 can vary from about 2 seconds to 10 seconds. These time periods are adjustable as well.

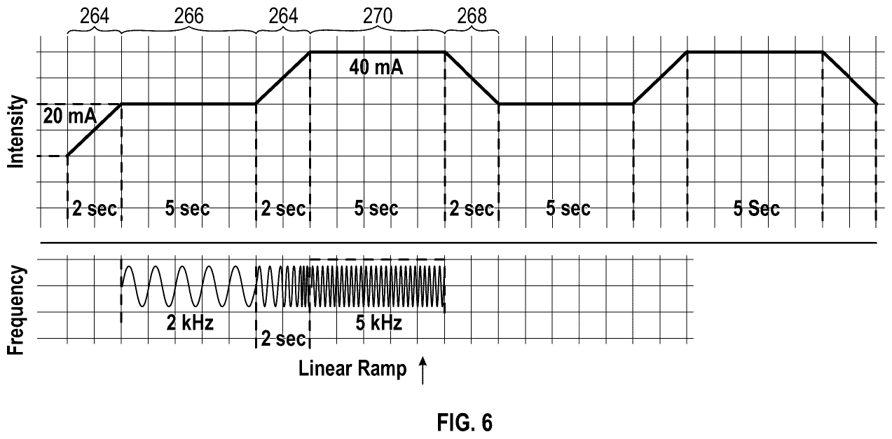

FIG. 6 is a graph illustrating another example intensity per time operation of the apparatus 100 for variable stimulation therapy, according to an example operation. In FIG. 6, the example therapy is modified as compared to the therapy shown in FIG. 5. For example, in FIG. 6, the cycle of stimulation treatment includes the first time period with the rise time 264 and the stimulatory treatment phase 266 as in the therapy of FIG. 5, so that the processor 106 implements a first frequency modulation (e.g., 2 KHz) coupled with a first increasing power modulation (e.g., 0-20 mA) so as to cause a muscle contraction level for the stimulatory treatment phase 266. The therapy in FIG. 6 includes the second time period with a second instance of the rise time 264 and the rest treatment phase 270 where during the rest treatment phase 270, the processor 106 implements a second frequency modulation (e.g., 5 KHz) which is higher than the first frequency modulation (e.g., 2 KHz) with a second increasing power modulation (e.g., 40 mA) which is higher than the first increasing power modulation (e.g., 20 mA) so as to be below the muscle contraction level for the rest treatment phase 270.

In this example, during the rise time 264, a rate of increase in frequency is greater than or equal to a rate of increase in power. During the fall time 268, a rate of decrease in power is greater than or equal to a rate of decrease in frequency.

The therapy in FIG. 6 includes muscle stimulation during the stimulatory treatment phase 266 followed by no muscle stimulation during the rest treatment phase 270. The difference as compared to the therapy in FIG. 5, however, is that during the rest treatment phase 270, as the frequency is increased, the amplitude or power is also increased in order to maintain a slight sensation of a beat frequency to the patient. It is known that when frequency is increased, a phase duration decreases, and so more number of cycles occur in one period that causes stimulation to spread out over a target area. As a result, a density of application of the stimulation in the target area decreases, and the patient will not feel the stimulation. In FIG. 6, during the rest treatment phase 270, amplitude or power is increased in order to achieve a sensation level, but not high enough to achieve a muscle contraction. Sensory nerves have a lower threshold for sensation than motor nerves, and thus, an action potential will only be caused in the sensory nerves at the higher frequency and higher power level during the rest treatment phase 270, and no action potential will be caused in the motor nerves during the rest treatment phase 270.

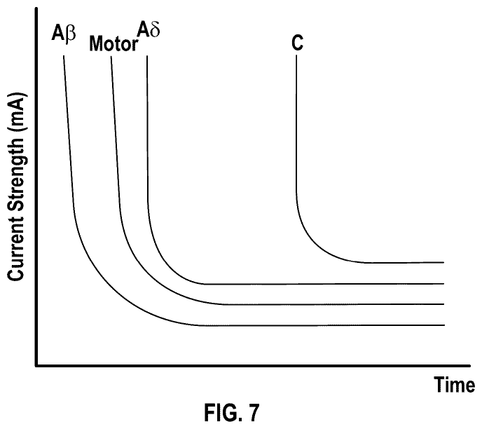

FIG. 7 is a graph illustrating power versus time for application of therapy to stimulate different nerves, according to an example implementation. In FIG. 7, sensory nerves Aβ have a lower threshold for sensation than the motor nerves, as shown in the graph with the Aβ nerves being triggered first by application of power. Additional sensory nerves are also shown to Aδ and C fibers that both have thresholds for sensation that require more time to activate. Thus, it is easier to stimulate sensory fibers Aβ than the motor nerves, and so a sensory stimulation can be achieved while not achieving a motor action potential, for example.

Referring back to FIG. 6, following the rest treatment phase 270, the therapy includes a fall time 268 and then the stimulatory treatment phase 266 and rest treatment phases 270 are repeated as shown.

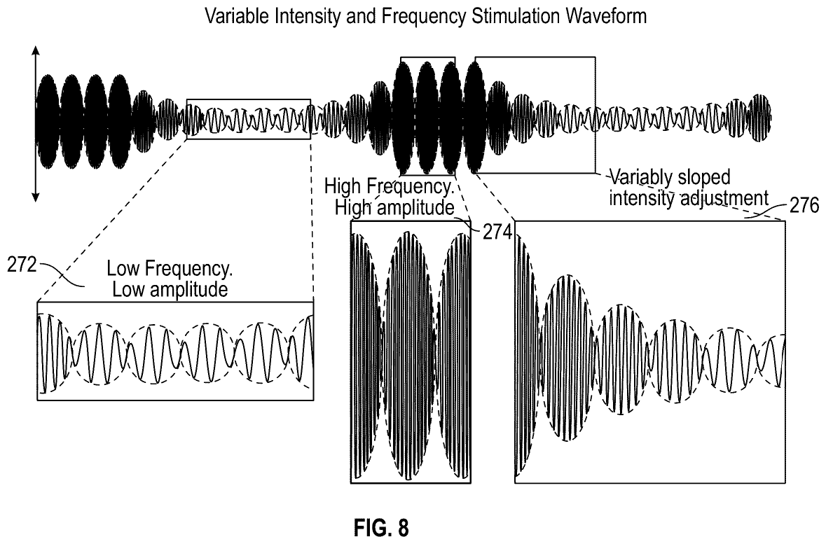

FIG. 8 is a graph illustrating intensity (e.g., amplitude or power) over time with variations in frequency, according to an example implementation. During the stimulatory treatment phase 266 in the therapy of FIG. 6, a low frequency and low amplitude signal 272 is generated and applied. During the rest treatment phase 270 in the therapy of FIG. 6, a high frequency and high amplitude signal 274 is generated and applied. During the rise time 264 and the fall time 268 in the therapy of FIG. 6, a variably sloped intensity signal 276 is generated and applied.

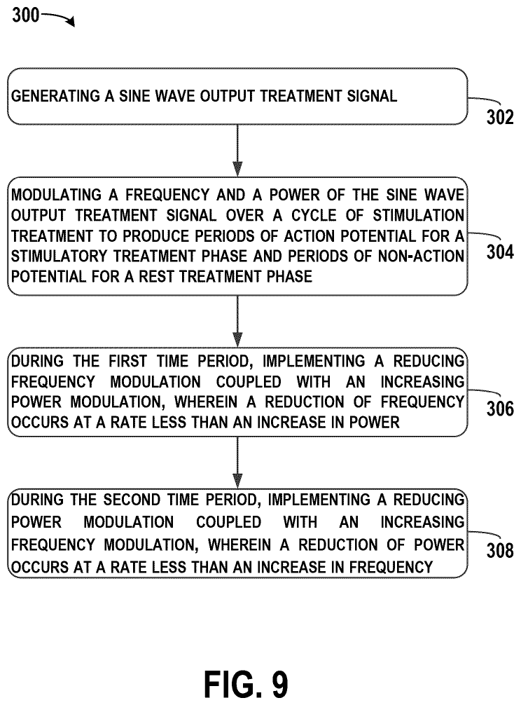

FIG. 9 shows a flowchart of an example of a method 300 of electro-medical stimulation applied by an apparatus, according to an example embodiment. The method 300 shown in FIG. 9 presents an example of a method that, for example, could be used or implemented by the apparatus 100 shown in FIG. 1, for example, and may be performed by components shown in FIGS. 2A-2B and FIG. 3. In some instances, components of the apparatus 100 are configured to perform the functions such that the components are structured (with hardware and/or software) to enable such performance. In other examples, components of the apparatus 100 are arranged to be adapted to, capable of, or suited for performing the functions, such as when operated in a specific manner. The method 300 includes one or more operations, functions, or actions as illustrated by one or more of blocks 302-308. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present embodiments. Alternative implementations are included within the scope of the example embodiments of the present disclosure in which functions may be executed out of order from that shown or discussed, including substantially concurrent or in reverse order, depending on the functionality involved, as would be understood by those reasonably skilled in the art.

At block 302, the method 300 includes generating a sine wave output treatment signal.

At block 304, the method 300 includes modulating a frequency and a power of the sine wave output treatment signal over a cycle of stimulation treatment to produce periods of action potential for a stimulatory treatment phase and periods of non-action potential for a rest treatment phase, and the cycle of stimulation treatment includes a first time period followed by a second time period.

At block 306, the method 300 includes during the first time period, implementing a reducing frequency modulation coupled with an increasing power modulation, and a reduction of frequency occurs at a rate less than an increase in power.

At block 308, the method 300 includes during the second time period, implementing a reducing power modulation coupled with an increasing frequency modulation, and a reduction of power occurs at a rate less than an increase in frequency.

In one example, the method 300 thus includes during the first time period, implementing the reducing frequency modulation simultaneously with the increasing power modulation, and during the second time period, implementing the reducing power modulation simultaneously with the increasing frequency modulation.

Depending on amounts of increase and decrease of the frequency and power, examples of the therapy include the cycle of stimulation treatment resulting in overall increases to the frequency and the power of the sine wave output treatment signal.

In some examples, the method 300 also includes repeating the cycle of stimulation treatment until a threshold upper frequency and upper power level is reached, and then reversing a direction of modulation of the frequency and the power of the sine wave output treatment signal during the first time period and the second time period until a threshold lower frequency and lower power level is reached. Reversing a direction of modulation includes changing an increase of a frequency or power to a decrease, or changing a decrease of a frequency or power to an increase, or both.

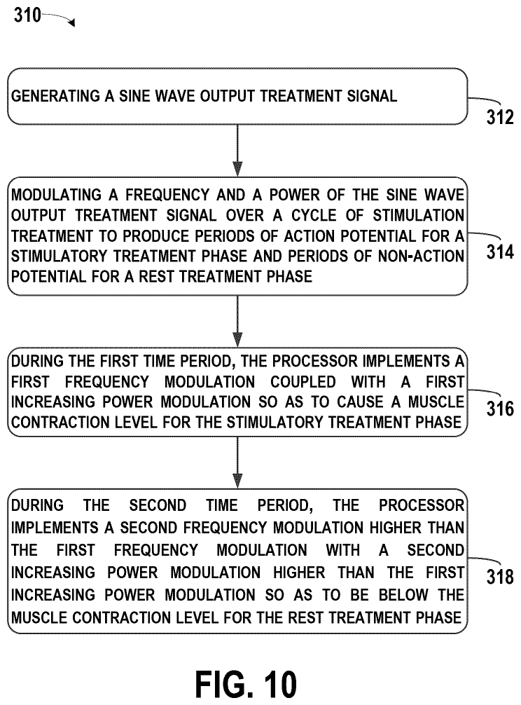

FIG. 10 shows a flowchart of another example of a method 310 of electro-medical stimulation applied by an apparatus, according to an example embodiment. The method 310 shown in FIG. 10 presents an example of a method that, for example, could be used or implemented by the apparatus 100 shown in FIG. 1, for example, and may be performed by components shown in FIGS. 2A-2B and FIG. 3. In some instances, components of the apparatus 100 are configured to perform the functions such that the components are structured (with hardware and/or software) to enable such performance. In other examples, components of the apparatus 100 are arranged to be adapted to, capable of, or suited for performing the functions, such as when operated in a specific manner. The method 310 includes one or more operations, functions, or actions as illustrated by one or more of blocks 312-318. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 312, the method 310 includes generating a sine wave output treatment signal.

At block 314, the method 310 includes modulating a frequency and a power of the sine wave output treatment signal over a cycle of stimulation treatment to produce periods of action potential for a stimulatory treatment phase and periods of non-action potential for a rest treatment phase, and the cycle of stimulation treatment includes a first time period followed by a second time period.

At block 316, the method 310 includes during the first time period, the processor implements a first frequency modulation coupled with a first increasing power modulation so as to cause a muscle contraction level for the stimulatory treatment phase.

At block 318, the method 310 includes during the second time period, the processor implements a second frequency modulation higher than the first frequency modulation with a second increasing power modulation higher than the first increasing power modulation so as to be below the muscle contraction level for the rest treatment phase.

Within examples, the method 310 includes the processor modulating the frequency between 1 KHz and 10 KHz, and the processor modulating the power between 0 Watts and 5 Watts.

By the term “approximate”, “about”, and/or the term “substantially”, it is meant that the recited characteristic, parameter, or value need not be achieved exactly, but that deviations or variations, including for example, tolerances, measurement error, measurement accuracy limitations and other factors known to those skilled in the art, may occur in amounts that do not preclude the effect the characteristic was intended to provide.

It should be emphasized that the above-described embodiments of the present disclosure are merely possible examples of implementations set forth for a clear understanding. Many variations and modifications may be made to the above-described embodiment(s) without departing substantially from the principles of the disclosure. All such modifications and variations are intended to be included herein within the scope of this disclosure.

What is claimed is:

1. An apparatus for electro-medical stimulation, the apparatus comprising:
a current generator to generate a sine wave output treatment signal; and
a processor coupled to the current generator to modulate a frequency and a power of the sine wave output treatment signal over a cycle of stimulation treatment to produce periods of action potential for a stimulatory treatment phase and periods of non-action potential for a rest treatment phase, wherein the cycle of stimulation treatment includes a first time period followed by a second time period, wherein:
during the first time period, the processor implements a reducing frequency modulation coupled with an increasing power modulation, wherein a reduction of frequency occurs at a rate less than an increase in power; and
during the second time period, the processor implements a reducing power modulation coupled with an increasing frequency modulation, wherein a reduction of power occurs at a rate less than an increase in frequency.

2. The apparatus of claim 1, wherein the first time period includes the processor implementing a ramp up of power during a rise time followed by holding power constant during the stimulatory treatment phase.

3. The apparatus of claim 2, wherein during the rise time, the reduction of frequency occurs.

4. The apparatus of claim 1, wherein the second time period includes the processor implementing a ramp down of power during a fall time followed by holding power constant during the rest treatment phase.

5. The apparatus of claim 4, wherein during the fall time, the increase in frequency occurs.

6. The apparatus of claim 1, wherein the cycle of stimulation treatment results in overall increases to the frequency and the power of the sine wave output treatment signal.

7. The apparatus of claim 1, wherein processor modulates the frequency between 1 KHz and 10 KHz.

8. The apparatus of claim 1, wherein the current generator generates the sine wave output treatment signal as a pre-modulated beat frequency signal having from 20 beats per second (BPS) to 250 BPS.

9. The apparatus of claim 1, wherein processor modulates the power between 0 Watts and 5 Watts.

10. The apparatus of claim 1, wherein the processor is configured to repeat the cycle of stimulation treatment until a threshold upper frequency and upper power level is reached, and then the processor is configured to reverse a direction of modulation of the frequency and the power of the sine wave output treatment signal during the first time period and the second time period until a threshold lower frequency and lower power level is reached.

11. The apparatus of claim 1, wherein:
during the first time period, the processor implements the reducing frequency modulation simultaneously with the increasing power modulation; and
during the second time period, the processor implements the reducing power modulation simultaneously with the increasing frequency modulation.

12. The apparatus of claim 1, wherein during the first time period, the cycle of stimulation treatment is in a period of action potential for the stimulatory treatment phase.

13. The apparatus of claim 1, wherein during the second time period, the cycle of stimulation treatment is in a period of non-action potential for the rest treatment phase.

14. The apparatus of claim 1, wherein the current generator comprises:
a pulse generator that generates digital signal pulses; and
a digital signal processor coupled to the pulse generator that processes the digital signal pulses to approximate a sine-wave-like output waveform.

15. A method of electro-medical stimulation applied by an apparatus, the method comprising:
generating a sine wave output treatment signal; and
modulating a frequency and a power of the sine wave output treatment signal over a cycle of stimulation treatment to produce periods of action potential for a stimulatory treatment phase and periods of non-action potential for a rest treatment phase, wherein the cycle of stimulation treatment includes a first time period followed by a second time period, wherein:
during the first time period, implementing a reducing frequency modulation coupled with an increasing power modulation, wherein a reduction of frequency occurs at a rate less than an increase in power; and
during the second time period, implementing a reducing power modulation coupled with an increasing frequency modulation, wherein a reduction of power occurs at a rate less than an increase in frequency.

16. The method of claim 15, wherein the cycle of stimulation treatment results in overall increases to the frequency and the power of the sine wave output treatment signal.

17. The method of claim 15, further comprising:
repeating the cycle of stimulation treatment until a threshold upper frequency and upper power level is reached, and then reversing a direction of modulation of the frequency and the power of the sine wave output treatment signal during the first time period and the second time period until a threshold lower frequency and lower power level is reached.

18. The method of claim 15, wherein:
during the first time period, implementing the reducing frequency modulation simultaneously with the increasing power modulation; and
during the second time period, implementing the reducing power modulation simultaneously with the increasing frequency modulation.

19. An apparatus for electro-medical stimulation, the apparatus comprising:
a current generator to generate a sine wave output treatment signal; and
a processor coupled to the current generator to modulate a frequency and a power of the sine wave output treatment signal over a cycle of stimulation treatment to produce periods of action potential for a stimulatory treatment phase and periods of non-action potential for a rest treatment phase, wherein the cycle of stimulation treatment includes a first time period followed by a second time period, wherein:
during the first time period, the processor implements a first frequency modulation coupled with a first increasing power modulation so as to cause a muscle contraction level for the stimulatory treatment phase;
during the second time period, the processor implements a second frequency modulation higher than the first frequency modulation with a second increasing power modulation higher than the first increasing power modulation so as to be below the muscle contraction level for the rest treatment phase.

20. The apparatus of claim 19, wherein processor modulates the frequency between 1 KHz and 10 KHz, and the processor modulates the power between 0 Watts and 5 Watts.

21. An apparatus for electro-medical stimulation, the apparatus comprising:
a current generator to generate a sine wave output treatment signal; and
a processor coupled to the current generator to modulate a frequency and a power of the sine wave output treatment signal over a cycle of stimulation treatment to produce periods of muscle contraction with motor nerve activation for a stimulatory treatment phase and periods of Aβ nerve-sensory stimulation for pain treatment for a rest treatment phase, wherein the cycle of stimulation treatment includes a first time period followed by a second time period, wherein:
during the first time period, the processor implements a reducing frequency modulation coupled with an increasing power modulation, wherein a reduction of frequency occurs at a rate less than an increase in power; and
during the second time period, the processor implements a reducing power modulation coupled with an increasing frequency modulation, wherein a reduction of power occurs at a rate less than an increase in frequency.

22. The apparatus of claim 21, wherein the first time period includes the processor implementing a ramp up of power during a rise time followed by holding power constant during the stimulatory treatment phase.

23. The apparatus of claim 22, wherein during the rise time, the reduction of frequency occurs.

24. The apparatus of claim 21, wherein the second time period includes the processor implementing a ramp down of power during a fall time followed by holding power constant during the rest treatment phase.

25. The apparatus of claim 24, wherein during the fall time, the increase in frequency occurs.

26. The apparatus of claim 21, wherein the cycle of stimulation treatment results in overall increases to the frequency and the power of the sine wave output treatment signal.

27. The apparatus of claim 21, wherein processor modulates the frequency between 1 KHz and 10 KHz.

28. The apparatus of claim 21, wherein the current generator generates the sine wave output treatment signal as a pre-modulated beat frequency signal having from 20 beats per second (BPS) to 250 BPS.

29. The apparatus of claim 21, wherein processor modulates the power between 0 Watts and 5 Watts.

30. The apparatus of claim 21, wherein the processor is configured to repeat the cycle of stimulation treatment until a threshold upper frequency and upper power level is reached, and then the processor is configured to reverse a direction of modulation of the frequency and the power of the sine wave output treatment signal during the first time period and the second time period until a threshold lower frequency and lower power level is reached.

31. The apparatus of claim 21, wherein:
during the first time period, the processor implements the reducing frequency modulation simultaneously with the increasing power modulation; and
during the second time period, the processor implements the reducing power modulation simultaneously with the increasing frequency modulation.

32. The apparatus of claim 21, wherein during the first time period, the cycle of stimulation treatment is in a period of muscle contraction with motor nerve activation for the stimulatory treatment phase.

33. The apparatus of claim 21, wherein during the second time period, the cycle of stimulation treatment is in a period of Aβ nerve-sensory stimulation for pain control for the rest treatment phase.

34. The apparatus of claim 21, wherein the current generator comprises:
a pulse generator that generates digital signal pulses; and
a digital signal processor coupled to the pulse generator that processes the digital signal pulses to approximate a sine-wave-like output waveform.

35. An apparatus for electro-medical stimulation, the apparatus comprising:
a current generator to generate a sine wave output treatment signal; and
a processor coupled to the current generator to modulate a frequency and a power of the sine wave output treatment signal over a cycle of stimulation treatment to produce periods of muscle contraction with motor nerve activation for a stimulatory treatment phase and periods of Aβ nerve-sensory stimulation for pain treatment for a rest treatment phase, wherein the cycle of stimulation treatment includes a first time period followed by a second time period, wherein:
during the first time period, the processor implements a first frequency modulation coupled with a first increasing power modulation so as to cause a muscle contraction level for the stimulatory treatment phase;
during the second time period, the processor implements a second frequency modulation higher than the first frequency modulation with a second increasing power modulation higher than the first increasing power modulation so as to be below the muscle contraction level for the rest treatment phase.
